(19) United States
(12) Patent Application Publication
(10) Pub. No.: US 2026/0036570 A1
(43) Pub. Date: Feb. 5, 2026

(54) BILIARY TRACT CANCER ORGANOID DERIVED FROM HUMAN CHEMICALLY-DERIVED HEPATIC PROGENITORS (HCDHS) USING CRISPR-CAS9

(71) Applicant: IUCF-HYU (INDUSTRY-UNIVERSITY COOPERATION FOUNDATION HANYANG UNIVERSITY), Seoul (KR)

(72) Inventors: Dongho CHOI, Seoul (KR); Ji Hyun SHIN, Seoul (KR); Junho HUR, Seoul (KR); Hyomin LEE, Seoul (KR); Michael ADISASMITA, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 19/286,328

(22) Filed: Jul. 31, 2025

(30) Foreign Application Priority Data
Aug. 2, 2024 (KR) 10-2024-0102835

(51) Int. Cl. G01N 33/50; C12N 5/09; C12N 9/22; C12N 15/11; C12N 15/90

(52) U.S. Cl. CPC G01N 33/5011; C12N 5/0693; C12N 9/226; C12N 15/11; C12N 15/907; G01N 33/5082; C12N 2310/20; C12N 2500/38; C12N 2501/11; C12N 2501/119; C12N 2501/12; C12N 2501/999; C12N 2533/90

(57) ABSTRACT

The present disclosure relates to a biliary tract cancer organoid and a method of producing the same. A new biliary tract cancer organoid system was developed in which CRISPR-Cas9 gene editing technology is applied to human chemically-derived hepatic progenitors to introduce mutations into some genes known as tumor suppressors or oncogenes, and the effects of the combined genetic mutations on human non-tumor, chemically-derived hepatic progenitors are analyzed to identify and screen the effects of the combined mutations on oncogenesis.

### CROSS-REFERENCE TO RELATED APPLICATION

This application claims priority to and the benefit of Korean Patent Application No. 2024-0102835, filed on Aug. 2, 2024, the disclosure of which is incorporated herein by reference in its entirety.

### INCORPORATION BY REFERENCE OF SEQUENCE LISTING

The instant application contains a Sequence Listing which has been filed electronically in xml format and is hereby incorporated by reference in its entirety. Said xml file, created on Jul. 29, 2025, is named Q311625_Sequence_Listing_ST26.xml as filed and is 8,295 bytes in size.

### BACKGROUND

### 1. Field of the Invention

The present invention relates to a biliary tract cancer organoid derived from human chemically-derived hepatic progenitors using CRISPR-Cas9 and a method of producing the same.

### 2. Discussion of Related Art

Biliary tract cancer (BTC) (bile duct cancer or cholangiocarcinoma) is a highly lethal form of hepatobiliary cancer, a refractory disease with no known therapy yet. To find new therapeutic methods for BTC, the development of a cancer modeling platform with high biological similarity to actual tumor tissue is required.

One emerging alternative for cancer disease modeling is cancer organoids. Patient-derived cancer organoids (PDOs) are generally derived from patient tumor samples used in clinical settings. However, the biliary tract tumor is known to be very small, and the relatively small size of tumor samples derived therefrom presents significant challenges in generating cancer organoids from these limited samples. Several studies have reported a low success rate of BTC organoid production, which is approximately 30%, and contamination with non-tumorous organoids is also prevalent during the PDO production process (Soito et al., Cell Reports, 2019).

Some studies have introduced specific genetic mutations by using gene-editing technologies such as CRISPR-Cas9 and analyzed the effects of single-gene mutations on cancer cells (Lo et al., Cancer Discov., 2021). However, single-gene mutations alone are difficult to induce cancer malignancy, and multiple mutations are required. Existing multi-gene combination screening using the CRISPR-Cas9 system has the limitation that, as the number of gene combinations increases, designing multiple single guide RNAs (sgRNAs) and Cas9 genes to deliver the gene combinations to target cells becomes complex and inefficient.

Therefore, there is an urgent need to develop new organoids for modeling BTC that can overcome the problems of limited cell supply and the limitations of gene editing technologies.

### RELATED ART DOCUMENTS

### Patent Document

(Patent Document 1) KR 10-2020-0075260 A

(Patent Document 2) KR 10-2018-0130625 A

### Non-Patent Document

(Non-Patent Document 1) Lee J E, et al. Bioeng Transl Med. 2022; 7:e10252.

### SUMMARY OF THE INVENTION

The present inventors have diligently studied and developed methods to overcome the limitations of existing biliary tract cancer (BTC) modeling.

As a result, by applying the CRISPR-Cas9 gene editing technology to chemically-derived human hepatic progenitors, mutations were introduced into some genes known as tumor suppressors or oncogenes. The effects of these recombinant genetic mutations on non-tumorous, chemically-derived human hepatic progenitors were analyzed to develop new organoids capable of elucidating the impact of these combined mutations on oncogenesis, thereby completing the present invention.

Therefore, an object of the present invention is to provide a BTC organoid capable of establishing a BTC-inducing environment with a short production period and limited sample size, and a method of producing the same

Another object of the present invention is to provide a use of the organoids in biliary tract disease treatment, disease modeling, drug screening, and the like.

To achieve the above-described objects, the present invention provides a method of producing a biliary tract cancer organoid, including: a step of culturing human primary hepatocytes (hPHs) in a reprogramming medium containing hepatocyte growth factor (HGF), A83-01, and CHIR99021 as reprogramming factors to reprogram them into human chemically-derived hepatic progenitors (hCdHs);
a step of reprogramming by inducing loss of function of TP53and BAP1 genes of hCdHs based on a clustered regularly interspaced short palindromic repeats (CRISPR)-CRISPR-associated nuclease 9 (Cas9) system containing a CRISPR-Cas9 guide RNA (gRNA); and
a step of subculturing the hCdHs reprogrammed in the above step in a reprogramming medium containing HGF, CHIR99021, and A83-01 to form a biliary tract cancer organoid.

The term “organoid” as used herein refers to a three-dimensional cell aggregate formed through self-renewal and self-organization derived from hepatic progenitors chemically induced from human primary hepatocytes (hPHs), and by re-aggregating and recombining the cells using a three-dimensional culture method, it can similarly reproduce physiologically active functions of the human body, including specific cells of an organ model, and can also be utilized as a cell therapy.

According to the method of producing a biliary tract cancer organoid of the present invention, hPHs are reprogrammed into hepatic progenitors through combined treatment with small molecules A83-01, CHIR99021, and HGF, and the expression of tumor suppression-related genes is regulated based on the CRISPR-Cas9 system, so that high proliferation potential and the ability to differentiate into hepatocytes and biliary epithelial cells are exhibited both in vitro and in vivo, thereby enabling the generation of patient-specific hepatic progenitors and providing a platform for personalized and stem cell-based regenerative medicine.

An organoid formed by introducing mutations in TP53 and BAP1 genes from non-tumor, chemically-derived hepatic progenitors according to the method of producing a biliary tract cancer organoid of the present invention has the advantage of enabling rapid identification and screening of effects on oncogenesis.

Each step of the method of producing the biliary tract cancer organoid of the present invention is described in detail below.

The first step is for reprogramming hPHs into hCdHs using HGF (H), an anaplastic lymphoma kinase (ALK) inhibitor A83-01 (A), and a glycogen synthase kinase (GSK) 3 inhibitor CHIR99021(C).

Reprogramming hPHs into hepatic progenitors includes a step of isolating hPHs from the livers of normal subjects and liver disease patients using a conventional two-step collagenase perfusion method; and a step of culturing the hPHs in a medium composition containing HGF, A83-01, and CHIR99021 to derive hepatic progenitors.

In the step of isolating hPHs, after treating hPHs with two different chemical agents, homologous polygonal cells appear and grow rapidly for several days, while coexisting human primary hepatocytes die. These chemically derived cells express genes of the hepatic and biliary epithelial lineage and are stained with a hepatic progenitor-specific marker. According to one embodiment of the present invention, the hepatic progenitors exhibited at least a 1.5-fold increase in expression of hepatic progenitor-specific markers including AFP, SOX9, ITGA6, HNF6, EpCAM, FOXJ1, HNF1B, CK19, CD44, and CD90 compared to hPHs.

Next-generation sequencing (NGS) studies have shown that rapidly growing CdHs exhibit a gene expression pattern similar to that of human hepatoblasts. CdHs derived from human livers may differentiate into hepatocytes and biliary epithelial cells, suggesting that they have the characteristics of bipotent hepatic stem cells. Even after 10 passages, CdHs retain their growth pattern and hepatic progenitor phenotype. CdHs also engraft and function for several weeks in an immunosuppressed mouse model following intrasplenic transplantation.

In the present invention, the reprogramming medium for reprogramming hPHs into hepatic progenitors includes HGF, A83-01, and CHIR99021, and HGF may be contained in the hPH reprogramming medium at a concentration of 2 to 100 ng/mL. When the concentration exceeds 100 ng/ml, apoptosis is induced, and when the concentration is less than 2 ng/mL, hepatic progenitors are not generated.

A83-01 is known as an ALK inhibitor, an inhibitor of transforming growth factor (TGF)-beta signaling and may be contained in the hPH reprogramming medium at a concentration of 0.4 to 4 μM. When the concentration exceeds 4 μM, apoptosis is induced, and when the concentration is less than 0.4 μM, the generation of hepatic progenitors is minimal.

CHIR99021 is known as a small molecule GSK-3 inhibitor and may be contained in the hPH reprogramming medium at a concentration of 0.3 to 3 μM. When the concentration exceeds 3 μM, apoptosis is induced, and when the concentration is less than 0.3 μM, the generation of hepatic progenitors is minimal.

The HGF, A83-01, and CHIR99021 may be added to a hepatocyte reprogramming medium. Therefore, the medium of the present invention may include a hepatocyte reprogramming medium and a growth medium.

The hepatocyte reprogramming medium may include any medium commonly used in not only somatic cell culture but also stem cell and progenitor culture. For example, it may include Dulbecco's Modified Eagle's Medium (DMEM), Minimal essential medium (MEM), Basal medium eagle (BME), RPMI1640, F-10, F-12, α-Minimal essential medium (α-MEM), Glasgow's Minimal essential medium (GMEM), and Iscove's Modified Dulbecco's Medium (IMDM). The medium used for culture generally contains a carbon source, a nitrogen source, and trace element components. For example, DMEM/F-12 medium supplemented with 0.1 μM dexamethasone, 10 mM nicotinamide, 1% insulin-transferrin-selenium (ITS) premix, and penicillin/streptomycin/glutamine was used, and other components necessary for cell culture may be contained without limitation.

hPHs may be cultured for 3 to 14 days in the reprogramming medium for reprogramming hPHs into hepatic progenitors to derive hepatic progenitors. When the culture time is outside the above-described range, hepatic progenitors may not be generated.

According to one embodiment, when culture was performed for 3 to 14 days in DMEM/F-12 medium supplemented with 10% fetal bovine serum (FBS), 0.1 μM dexamethasone, 10 mM nicotinamide, 1% ITS premix, penicillin/streptomycin/glutamine, 20 ng/mL epidermal growth factor (EGF), 20 ng/mL HGF, 4 μM A-83-01, and 3 μM CHIR99021, hepatic progenitors were derived, and the characteristics of hepatic progenitors, that is, the expression of hepatic progenitor-specific markers, began to increase from day 7 and were maintained for 14 days or more.

The hepatic progenitors derived from hPHs in the reprogramming medium for reprogramming hPHs into hepatic progenitors are cells exhibiting a higher expression level of hepatic progenitor-specific markers compared to hPHs and having the characteristics of bipotent stem cells that differentiate into hepatocytes and biliary epithelial cells.

Therefore, through the reprogramming step, hepatic progenitors that exhibit at least a 1.5-fold increase in expression levels of hepatic progenitor-specific markers including AFP, SOX9, ITGA6, HNF6, EpCAM, FOXJI, HNF1B, CK19, CD44, and CD90 compared to hPHs and have bipotent stem cell characteristics that differentiate into hepatocytes and biliary epithelial cells can be obtained, and the hepatic progenitors can be provided as a source of hepatic progenitors for liver regeneration due to their bipotent stem cell characteristics.

In the method of producing a biliary tract cancer organoid of the present invention, the second step is a step of reprogramming by inducing loss of function of the TP53 and BAP1 genes of hCdHs based on a CRISPR-Cas9 system containing CRISPR-Cas9 gRNA.

In the present invention, the CRISPR-Cas9 guide RNA for the TP53 gene may be one or more selected from the group consisting of SEQ ID NO: 1, SEQ ID NO: 2, SEQ ID NO: 3, and SEQ ID NO: 4 but is not limited thereto.

In the present invention, the CRISPR-Cas9 guide RNA for the BAP1 gene may be one or more selected from the group consisting of SEQ ID NO: 5, SEQ ID NO: 6, SEQ ID NO: 7, and SEQ ID NO: 8 but is not limited thereto.

A carrier of the CRISPR-Cas9 guide RNA may be one or more selected from the group consisting of a viral vector, a plasmid vector, an Agrobacterium vector, and an RNA-protein complex (ribonucleoprotein complex) but is not limited thereto.

The term “guide RNA” as used herein refers to RNA specific to target DNA, capable of forming a complex with the Cas9 protein, and RNA that brings the Cas9 protein to the target DNA. RNA may be delivered to a cell or an organism in the form of RNA or in the form of DNA encoding the guide RNA. In addition, the guide RNA may be in the form of isolated RNA or in the form of RNA contained in a viral vector or encoded within a vector.

In the present invention, the guide RNA may be a dual RNA including a CRISPR RNA (crRNA) and a transactivating crRNA (tracrRNA) that hybridize with the target DNA, or a single-stranded guide RNA (sgRNA) that includes portions of the crRNA and tracrRNA and hybridizes with the target DNA. In one embodiment of the present invention, the guide RNA is a sgRNA. Any guide RNA may be used in the present invention as long as it includes essential portions of the crRNA and tracrRNA and a portion complementary to the target.

The term “5′-truncated guide RNA” used in referring to the CRISPR-Cas9 system-based genome editing is an sgRNA including a crRNA in which two nucleotides are deleted from the 5′-end of a guide RNA including a nucleotide sequence complementary to the target DNA, and the term is used interchangeably herein with 5′-truncated sgRNA.

The term “TP53” used herein is the most widely known gene involved in human tumorigenesis, and it regulates the cell cycle by inducing apoptosis. TP53 suppresses tumorigenesis by inhibiting the proliferation of cells with damaged DNA or inducing apoptosis in cells with irreparable damage. When mutations occur due to the problems in the TP53 gene, various types of cancer may develop.

The term “BRCA1 associated protein 1 (BAP1)” as used herein refers to a member of the ubiquitin C-terminal hydrolase subfamily of deubiquitinating enzymes involved in removing ubiquitin from proteins, and the enzyme expressed by the encoding gene binds to the really interesting new gene (RING) finger domain of the breast cancer type 1 susceptibility protein (BRCA1) and serves as a tumor suppressor. BAP1 is involved in transcriptional regulation, cell cycle and growth regulation, responses to DNA damage, and chromatin dynamics.

The terms “nucleic acid sequence,” “nucleotide sequence,” and “polynucleotide sequence” as used herein refer to oligonucleotides or polynucleotides, and fragments or portions thereof, and DNA or RNA of a genomic or synthetic origin, which may be single-stranded or double-stranded, and represent either a sense strand or an antisense strand.

The term “edit,” “editing,” or “edited” as used herein refers to a method of altering the nucleic acid sequence of a polynucleotide (e.g., a wild-type naturally occurring nucleic acid sequence or a mutated naturally occurring sequence) by selectively deleting a specific genomic target or incorporating a new specific sequence using an exogenously supplied DNA template. Such specific genomic targets may include a chromosomal region, mitochondrial DNA, a gene, a promoter, an open reading frame, or any nucleic acid sequence but are not limited thereto.

In the method of producing the biliary tract cancer organoid of the present invention, the third step is a step of forming a biliary tract cancer organoid by subculturing the reprogrammed hCdHs in a reprogramming medium while suppressing gene expression, and then culturing the same in a biliary tract cancer organoid culture medium.

The subculture may be performed by culturing human hepatic progenitors in a reprogramming medium for one to three passages and mixing the human hepatic progenitors and the reprogramming medium in a volume ratio of 1:3 to 1:5 every five days.

Specifically, the biliary tract cancer organoid may proliferate during 10 to 20 passages and maintain the characteristics and functions of mature hepatic cells. In other words, the biliary tract cancer organoid is a proliferative organoid.

In the present invention, the method of producing the biliary tract cancer organoid further includes a step of culturing the formed biliary tract cancer organoid. In the present invention, a biliary tract cancer organoid culture medium may include one or more selected from the group consisting of N-2 supplement, B27 supplement, N-acetyl cysteine, gastrin, R-spondin 1 (RSPO1), fibroblast growth factor 10 (FGF10), HGF, nicotinamide, 5 μM of A83-01, and 10 μM of forskolin (FSK), but is not limited thereto.

In the present invention, the step of culturing the biliary tract cancer organoid includes a step of sequentially culturing an initial biliary tract cancer organoid and a step of culturing a second biliary tract cancer organoid.

The step of culturing the initial biliary tract cancer organoid may include a step of amplifying a Wnt signaling pathway involved in a cell proliferation signaling pathway.

In the present invention, the initial biliary tract cancer organoid culture medium may additionally include one or more selected from the group consisting of Wnt3a, Noggin, and Y27632, but is not limited thereto.

In the present invention, the step of culturing the initial biliary tract cancer organoid may include culturing the initial biliary tract cancer organoid within a Matrigel dome for three days, but is not limited thereto.

In the present invention, the step of culturing the second biliary tract cancer organoid may include culturing the biliary tract cancer organoid formed from the initial biliary tract cancer organoid for 4 to 14 days to mature the biliary tract cancer organoid, but is not limited thereto.

Another aspect of the present invention is a biliary tract cancer organoid capable of establishing a biliary tract cancer environment with a short production period and limited sample size.

In one embodiment of the present invention, the biliary tract cancer organoid with TP53/BAP1 knockout exhibited a morphologically distinct phenotype when compared with normal hCdHs organoids without TP53/BAP1 knockout, showing a darker and thicker shape with multiple luminal structures. In addition, the organoid exhibited morphological characteristics very similar to those derived from the SNU1196 biliary tract cancer cell line (SNU1196_O) and the cancer organoid derived from an extrahepatic biliary tract cancer patient (eCC_PDO) (FIG. 2B).

In one embodiment of the present invention, as a result of hematoxylin and eosin (H&E) staining and pathological analysis, the biliary tract cancer organoid exhibited characteristics of malignant organoids derived from biliary cells. In addition, periodic acid-Schiff (PAS) and Alcian blue staining showed mucin (FIG. 2C).

In one embodiment of the present invention, the biliary tract cancer organoid expressed markers associated with biliary epithelial cells, such as SOX4 (intrahepatic biliary epithelial cells), S100P (perihilar cholangiocarcinoma), SOX17 (extrahepatic biliary epithelial cells), and TFF3 (gallbladder) (FIG. 2D). In addition, the organoid lumen exhibited overexpression of MUCI and changes in organoid polarity due to EpCAM expression (FIG. 2E).

The biliary tract cancer organoid of the present invention is produced by reprogramming human chemically-derived hepatic progenitors (hCdHs), obtained by chemically reprogramming human primary hepatocytes (hPHs) using HGF, A83-01, and CHIR99021, by inducing loss of function of the TP53 and BAP1 genes using the CRISPR-Cas9 system, and then subculturing the reprogrammed hCdHs in a reprogramming medium containing HGF, CHIR99021, and A83-01. The biliary tract cancer organoid of the present invention can overcome the disadvantage of conventional biliary tract cancer screening, which takes a long time, and can establish a biliary tract cancer-inducing environment in a short period of time at a low cost and provide a screening environment for biliary tract cancer treatment.

In one embodiment, the size of the biliary tract cancer organoid may be 100 to 400 μm.

Another aspect of the present invention relates to a method of screening for the prognosis of biliary tract cancer, including a step of measuring cell viability in a biliary tract cancer organoid.

The test substance is a substance expected to exhibit an ameliorating or therapeutic effect in a biliary tract disease according to a conventional selection method and may include, for example, any substance, molecule, element, compound, entity, or a combination thereof. For example, it may include a protein, a polypeptide, a small organic molecule, a polysaccharide, a polynucleotide, and the like. In addition, it may be a natural product, a synthetic compound, a chemical compound, or a combination of two or more substances.

In the present invention, a method of screening biliary toxic drugs may be performed by treating a biliary tract cancer organoid with a test substance and measuring cell viability, thereby comparing the measured cell viability with a control group or human biliary tract cell that is not treated with the test substance.

Specifically, in a case in which cell viability decreases when a biliary tract cancer organoid is treated with a test substance in a differentiation medium, the test substance is determined to be a biliary toxic substance. For example, as a result of comparing the sensitivity and accuracy of toxic drugs to the control group through cell viability, the biliary tract cancer organoid may be confirmed to have significantly higher sensitivity and accuracy.

Another aspect of the present invention relates to a method of screening drugs for ameliorating or treating biliary tract cancer, including: a step of producing a biliary tract cancer organoid model of biliary tract cancer by bringing a biliary tract cancer organoid into contact with a biliary tract cancer-inducing substance; and a step of measuring the viability of the organoid after bringing the test substance into contact with the biliary tract cancer organoid model of biliary tract cancer and comparing the measured viability with the viability of a biliary tract cancer organoid model of biliary tract cancer that is not brought into contact with the test substance.

In the present invention, the biliary tract cancer-inducing substance is a substance that induces biliary tract cancer in a biliary tract cancer organoid, and may include, for example, any substance, molecule, element, compound, entity, or a combination thereof. For example, it may include a protein, a polypeptide, a small organic molecule, a polysaccharide, a polynucleotide, and the like. In addition, it may be a natural product, a synthetic compound, a chemical compound, or a combination of two or more substances.

In the present invention, a method of screening drugs for ameliorating or treating biliary tract cancer may be performed by treating the biliary tract cancer organoid with a test substance, measuring cell viability, and comparing the measured cell viability with a control group or adult biliary tract cell that is not treated with the test substance.

The advantages and features of the present invention and the methods for achieving them will become clearer with reference to the embodiments described in detail below. However, the present invention is not limited to the embodiments disclosed below but may be implemented in various different forms. These embodiments are provided solely to ensure that the disclosure of the present invention is complete and to fully inform those skilled in the art of the scope of the invention. The present invention is defined solely by the scope of the claims.

The present invention relates to a biliary tract cancer organoid and a method of producing the same. In the present invention, CRISPR-Cas9 gene editing technology is applied to human chemically-derived hepatic progenitors to introduce mutations into some genes known as tumor suppressors or oncogenes, and the effects of the combined genetic mutations on human non-tumor, chemically-derived hepatic progenitors are analyzed to identify and screen the effects of the combined mutations on oncogenesis. The biliary tract cancer organoid of the present invention can overcome the disadvantage of the conventional biliary tract cancer screening, which takes a long time, and can establish a biliary tract cancer-inducing environment in a short period of time at a low cost and provide a screening environment for biliary tract cancer treatment.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1A is a diagram illustrating a process of transforming hCdHs with plasmid-mediated CRISPR-Cas9 according to one embodiment of the present invention;

FIG. 1B is a diagram illustrating a design process of a guide RNA targeting TP53 exons 3 and 4 and BAP1 exons 3 and 4 according to one embodiment of the present invention;

FIG. 1C is a diagram illustrating the results of observing TP53^{KO}, BAP1^{KO}, and TP53-BAP1^{DKO} (double knockout) cell lines produced through gene editing according to one embodiment of the present invention;

FIG. 1D is a diagram illustrating the results of measuring the expression of each gene using reverse transcription polymerase chain reaction (RT-PCR) according to one embodiment of the present invention;

FIG. 2A is a diagram illustrating the process of producing a biliary tract cancer organoid by culturing the hCdH TP53/BAP1D^{KO} cell line in a Matrigel dome according to one embodiment of the present invention;

FIG. 2B is a diagram illustrating the results of comparing a biliary tract cancer organoid (hCdHO_DKO) according to one embodiment of the present invention with a conventional biliary tract cancer organoid model (SNU1196-O, eCC_PDO); and

FIG. 2C is a diagram illustrating the results of periodic acid-Schiff (PAS) and Alcian blue staining to confirm the presence of mucin according to one embodiment of the present invention;

FIG. 2D shows a graph illustrating the differentiation of SOX4 and TFF3, which are markers associated with biliary epithelial differentiation, in a biliary tract cancer organoid according to one embodiment of the present invention; and

FIG. 2E is a diagram illustrating the results of observing changes in organoid polarity due to MUC1 overexpression and EpCAM expression in the organoid lumen according to one embodiment of the present invention.

### DETAILED DESCRIPTION OF EXEMPLARY EMBODIMENTS

Hereinafter, one or more embodiments will be described in more detail through examples. However, these examples are intended to exemplify one or more embodiments, and the scope of the present invention is not limited to these examples.

### Terms

To ensure a clear understanding of the present invention, the following terms are defined:

Human hepatocytes: hPHs; human chemically-derived hepatic progenitors: hCdHs

Clustered regularly interspaced short palindromic repeats (CRISPR)-CRISPR-associated nuclease 9: CRISPR-Cas9

Biliary tract cancer organoid: human chemically-derived progenitor (hCdH)-derived biliary tract cancer organoid (hCdH-BTCO)

### Manufacturing Example 1

### Establishment of Chemically-Derived Hepatic Progenitors

The chemically-derived hepatic progenitors (hCdHs) disclosed herein are derived from human hepatic progenitors, and they were produced as described in a recent publication (Buisson E M, Park S H, Kim M, Kang K, Yoon S, Lee J E, et al. Transplantation of patient-specific bile duct bioengineered with chemically reprogrammed and microtopographically differentiated cells. Bioeng Transl Med. 2022; 7:e10252.).

Specifically, after culturing in William's medium for 24 hours, human primary hepatocytes (hPHs) were cultured in Dulbecco's Modified Eagle's Medium (DMEM)/F-12 (11965, Gibco, CA, USA) supplemented with 10 mM nicotinamide (Sigma-Aldrich, MO, USA), 1% penicillin/streptomycin (Gibco), 20 ng/mL hepatocyte growth factor (HGF) (Peprotech, NJ, USA), 20 ng/mL epidermal growth factor (EGF) (Peprotech), 4 μM A83-01 (Gibco), and 3 μM CHIR99021 (StemCell Technologies) in an incubator under the conditions of 37° C. and CO₂.

### Example 1

### Multiplex Genome Editing Via CRISPR-Cas9

### 1-1. CRISPR-Cas9

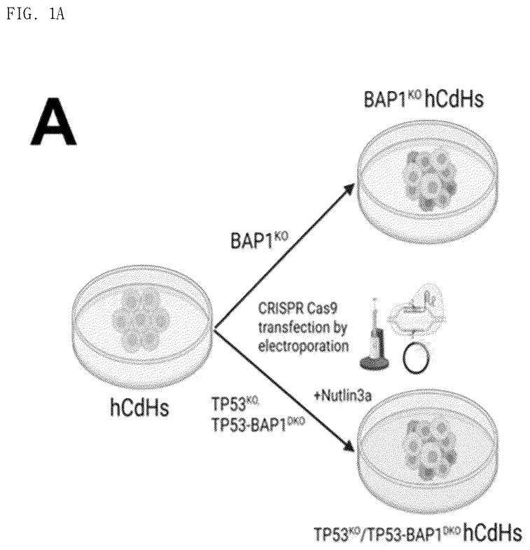

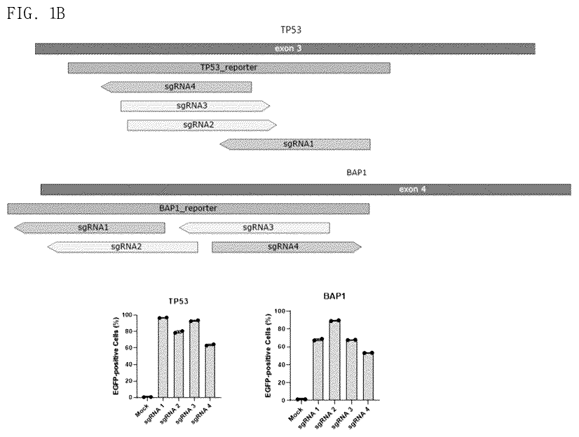

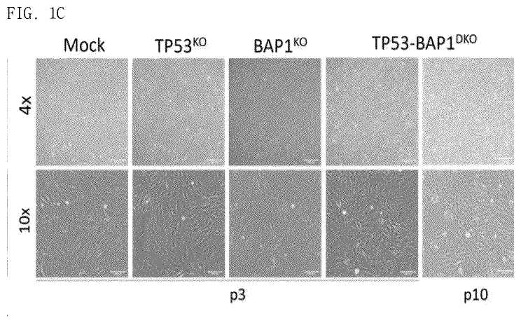

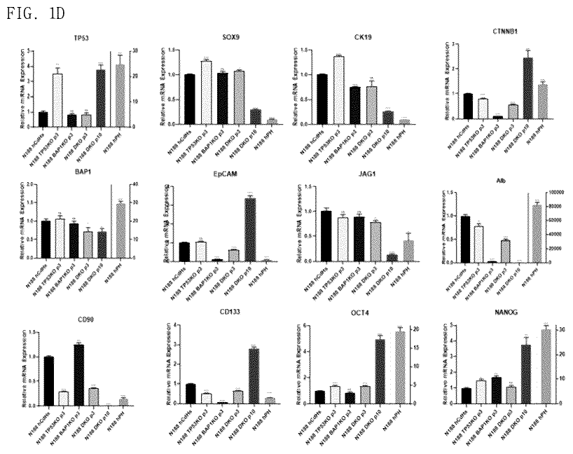

hCdHs lacking TP53 and BAP1 were generated from hPHs through plasmid-mediated CRISPR-Cas9 transfection (FIG. 1A).

1-2. Construction and Verification of sgRNA Expression Plasmid

Four gRNAs were designed for each gene, targeting exons 3 and 4 of each of the TP53 and BAP1 genes (FIG. 1B). To construct and verify the sgRNA expression plasmids, knockout (KO) of the relevant genes was first confirmed in HEK293T cells.

The cells were treated with 2.5 μg/μl of an sgRNA expression plasmid capable of knocking out TP53 and BAP1, 5 μg/μl of a Cas9 expression plasmid, and 2.5 μg/μl of a reporter plasmid containing green and red fluorescent proteins. Cells were cultured at 37° C. for one to two days.

The reporter plasmid is capable of reflecting transfection and gene editing effects. Since the plasmid may constitutively express the red fluorescent protein, it is possible to confirm only the transfected cells. For example, when gene editing occurs, a switch from the red fluorescent protein to the green fluorescent protein occurs. Therefore, this system is capable of isolating only cells expressing the green fluorescent protein so that only mutant cells are isolated.

After the transfection attempt, to screen only transfected cells, cells expressing the red fluorescent protein were first isolated using fluorescence-activated cell sorting (FACS).

To verify the gene editing effect of the sgRNA actually produced in these isolated cells, only cells in which the original red fluorescent protein had switched to the green fluorescent protein were isolated through FACS, and the gene editing effect was analyzed.

As a result, four subsequent cell lines, which were a mock cell line, TP53^{KO}, BAP1^{KO}, and TP53/BAP1^{DKO}, were established (FIG. 1C).

### 1-3. Quantitative PCR Analysis

The cellular transcriptome profile was analyzed using quantitative RT-PCR (FIG. 1D). To analyze the cellular transcriptome using quantitative RT-PCR, 1×10⁵ to 1×10⁶ cells were collected from each group, and RT-PCR was performed through the following three-step process.

Step 1: To purify only the cellular transcriptome (RNA), cells were treated with TRIzol, chloroform, and DNase to lyse the cell membrane. Thereafter, pure RNA was extracted and purified through centrifugation and treatment with 70% ethanol.

Step 2: To obtain only mRNA information from the purified RNA, a reverse transcriptase, dNTPs, a reaction buffer, dTT oligonucleotides, and an RNase inhibitor were used to induce complementary DNA (cDNA) synthesis from the mRNA. As a result, cDNA containing only mRNA-related information was obtained.

Step 3: The obtained cDNA was added to a master mix solution containing primers for the genes of interest, polymerase, and materials needed for PCR. The resulting mixture was plated onto a 96-well plate and placed in an RT-PCR analyzer for analysis.

The experimental results showed significant changes in gene expression profiles, particularly in the TP53/BAP1^{DKO} cell line, depending on the number of passages (p10). These changes were characterized by upregulation of cancer stem cell markers and pluripotency genes (EpCAM, CD133, OCT4, NANOG), as well as a proliferation-related gene (CTNNB1) (FIG. 1D).

### Example 2

### Production of Cancer Organoid

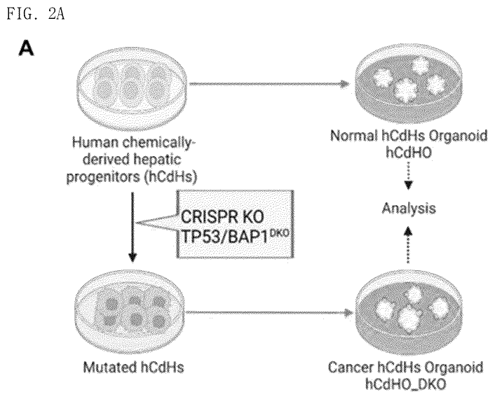

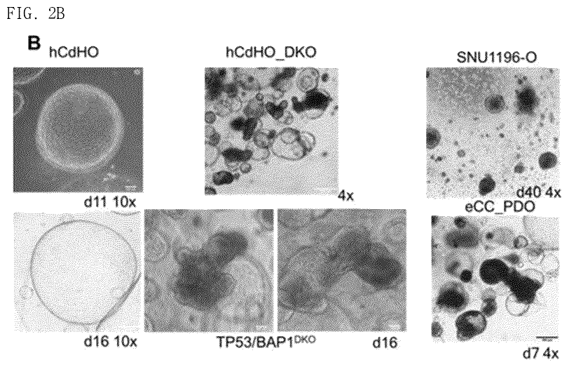

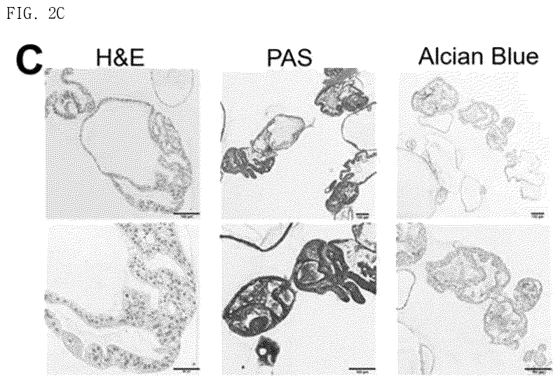

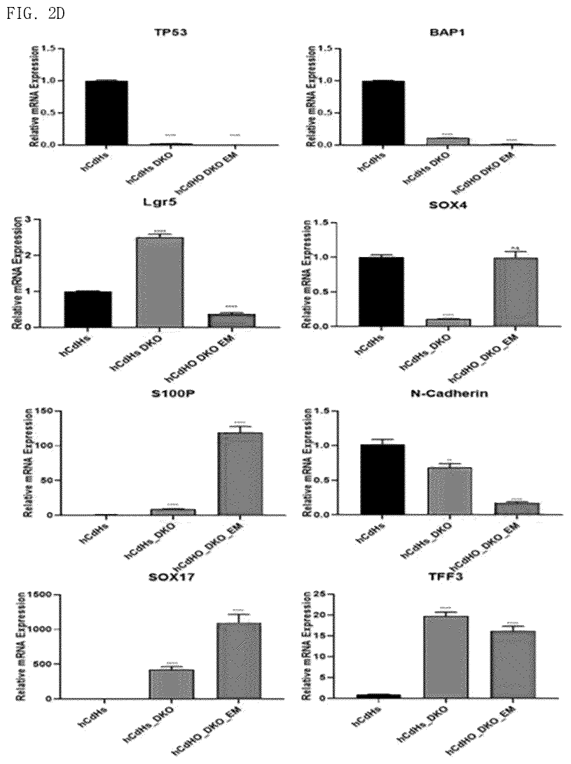

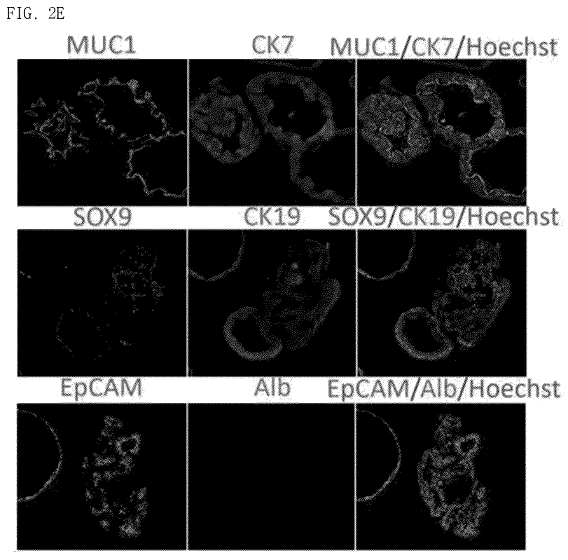

A cancer organoid was produced in a Matrigel dome using the hCdH TP53/BAP1^{DKO} cell line (FIG. 2A). Reprogrammed hepatic progenitors (hCdHs) with double knockout of TP53 and BAP1 were passaged up to three times in a reprogramming medium containing HGF, CHIR99021, and A83-01, and then 2 ml of TripLE solution was added to detach the cells from the culture dish. Thereafter, 5 ml of the culture medium was transferred to a 15 ml tube and centrifuged at 1200 rpm and 4° C. for five minutes.

After centrifugation, the remaining solution was removed, leaving only the generated cells. 70 μl of a Matrigel solution was added and mixed with 1×10⁴ cells. Thereafter, only 50 μl of Matrigel solution was dispensed into the center of a 24-well plate and placed in a 37° C. incubator for 10 minutes to form a Matrigel dome. To amplify the Wnt signaling pathway involved in the cell proliferation signaling pathway, cells were cultured for three days in an initial biliary tract cancer organoid culture medium containing advanced Dulbecco's Modified Eagle's Medium (adDMEM)/F12, Minimal essential medium (MEM), N-2 supplement (100×), B27 supplement (50×, serum free), N-acetyl cysteine (NAC), gastrin, R-spondin 1 (RSPO1), fibroblast growth factor 10 (FGF 10), nicotinamide (1×), forskolin (FSK), EGF, HGF, A83-01, Wnt3a 100 ng/ml, Noggin 25 ng/ml, Y27632 10 μM. Next, the biliary tract cancer organoid formed as the initial biliary tract cancer organoid was cultured for 4 to 14 days in a 24-well plate in a biliary tract cancer organoid culture medium lacking Wnt3a, Noggin, and Y27632.

Compared to normal hCdH organoids (hCdHO), hCdHO TP53/BAP^{DKO} (hCdHO_DKO) exhibited a markedly different morphological phenotype. The engineered cancer organoid exhibited malignant features, with a darker and thicker organoid that developed multiple luminal structures. The organoid was very similar to those derived from the SNU1196 biliary tract cancer cell line (SNU1196_O) and the cancer organoid derived from an extrahepatic biliary tract cancer patient (eCC_PDO) (FIG. 2B).

### Example 3

### Pathological Analysis

As a result of hematoxylin and eosin (H&E) staining and a pathological analysis, a malignant organoid derived from biliary cells exhibited significantly different characteristics from a non-malignant wild-type organoid, confirming the characteristics of a malignant organoid derived from biliary cells. In addition, PAS staining and Alcian blue staining confirmed the presence of mucin (FIG. 2C). In addition, complete knockout of the TP53 and BAP1 genes was confirmed at the mRNA level in the organoid culture (FIG. 2D). Furthermore, the organoid expressed markers associated with biliary epithelial cells, such as SOX4 (intrahepatic biliary epithelial cells), S100P (perihilar cholangiocarcinoma), SOX17 (extrahepatic biliary epithelial cells), and TFF3 (gallbladder) (FIG. 2D). In addition, changes in organoid polarity due to MUC1 overexpression and EpCAM expression were observed in the lumen of the organoid (FIG. 2E).

### LIST OF SEQUENCES

An electronic file including a list of sequences is attached.

What is claimed is:

1. A method of producing a biliary tract cancer organoid, comprising:
a step of culturing human primary hepatocytes (hPHs) in a reprogramming medium containing hepatocyte growth factor (HGF), A83-01, and CHIR99021 as reprogramming factors to reprogram the hPHs into human chemically-derived hepatic progenitors (hCdHs);
a step of reprogramming by inducing loss of function of TP53 and BAP1 genes of hCdHs based on a clustered regularly interspaced short palindromic repeats (CRISPR)-CRISPR-associated nuclease 9 (Cas9) system containing a CRISPR-Cas9 guide RNA (gRNA); and
a step of subculturing the hCdHs reprogrammed in the above step in a reprogramming medium containing HGF, CHIR99021, and A83-01 to form a biliary tract cancer organoid.

2. The method of claim 1, wherein the HGF is contained in the reprogramming medium at a concentration of 2 to 100 ng/ml.

3. The method of claim 1, wherein the A83-01 is contained in the reprogramming medium at a concentration of 0.4 to 4 μM.

4. The method of claim 1, wherein the CHIR99021 is contained in the reprogramming medium at a concentration of 0.3 to 3 μM.

5. The method of claim 1, wherein the hPHs are cultured for 3 to 14 days.

6. The method of claim 1, wherein the reprogramming medium is one or more selected from the group consisting of Dulbecco's Modified Eagle's Medium (DMEM), Minimal essential medium (MEM), Basal medium eagle (BME), RPMI1640, F-10, F-12, α-Minimal essential medium (α-MEM), Glasgow's Minimal essential medium (GMEM), and Iscove's Modified Dulbecco's Medium (IMDM).

7. The method of claim 1, wherein the CRISPR-Cas9 guide RNA for the TP53 gene is one or more selected from the group consisting of SEQ ID NO: 1, SEQ ID NO: 2, SEQ ID NO: 3, and SEQ ID NO: 4.

8. The method of claim 1, wherein the CRISPR-Cas9 guide RNA for the BAP1 gene is one or more selected from the group consisting of SEQ ID NO: 5, SEQ ID NO:
6, SEQ ID NO: 7, and SEQ ID NO: 8.

9. The method of claim 1, wherein the subculture is performed by culturing human hepatic progenitors in a reprogramming medium for one to three passages and mixing the human hepatic progenitors and the reprogramming medium in a volume ratio of 1:3 to 1:5 every five days.

10. A biliary tract cancer organoid produced according to the method of claim 1, capable of establishing a biliary tract cancer-inducing environment with a short production period and limited samples.

11. A method of screening for the prognosis of biliary tract cancer, comprising:
a step of bringing a test substance into contact with the biliary tract cancer organoid of claim 10; and
a step of measuring cell viability in the biliary tract cancer organoid.

12. A method of screening drugs for ameliorating or treating biliary tract cancer, comprising:
a step of producing a biliary tract cancer organoid model of biliary tract cancer by bringing the biliary tract cancer organoid of claim 10 into contact with a biliary tract cancer-inducing substance; and
a step of measuring viability of the organoid after bringing the test substance into contact with the biliary tract cancer organoid model of biliary tract cancer and comparing the measured viability with the viability of a biliary tract cancer organoid model of biliary tract cancer that is not brought into contact with the test substance.


SEQUENCE LISTING (8 sequences)

SEQ ID NO: 1 - DNA, 23 bp, synthetic construct
  source 1..23: mol_type = other DNA
cagaatgcaagaagcccagacgg

SEQ ID NO: 2 - DNA, 23 bp, synthetic construct
  source 1..23: mol_type = other DNA
ggcagctacggtttccgtctggg

SEQ ID NO: 3 - DNA, 23 bp, synthetic construct
  source 1..23: mol_type = other DNA
gggcagctacggtttccgtctgg

SEQ ID NO: 4 - DNA, 23 bp, synthetic construct
  source 1..23: mol_type = other DNA
gacggaaaccgtagctgccctgg

SEQ ID NO: 5 - DNA, 23 bp, synthetic construct
  source 1..23: mol_type = other DNA
gataaatccatatacagggctgg

SEQ ID NO: 6 - DNA, 23 bp, synthetic construct
  source 1..23: mol_type = other DNA
aggaagataaatccatatacagg

SEQ ID NO: 7 - DNA, 23 bp, synthetic construct
  source 1..23: mol_type = other DNA
gctcttcgatccatttgaacagg

SEQ ID NO: 8 - DNA, 23 bp, synthetic construct
  source 1..23: mol_type = other DNA
tcaaatggatcgaagagcgccgg